(19) United States
(12) Patent Application Publication
(10) Pub. No.: US 2011/0245642 A1
(43) Pub. Date: Oct. 6, 2011

(54) ELECTRONIC ENDOSCOPE SYSTEM

(76) Inventor: Yasuhiro MINETOMA, Kanagawa (JP)

(21) Appl. No.: 13/079,408

(22) Filed: Apr. 4, 2011

(30) Foreign Application Priority Data
Apr. 5, 2010 (JP) 2010-087061

(51) Int. Cl. A61B 5/1455

(52) U.S. Cl. 600/324

(57) ABSTRACT

An electronic endoscope system includes a light source device, an electronic endoscope for sequentially illuminating a subject tissue containing a blood vessel inside a body cavity with the light, and sequentially outputting image data of wavelength bands of the subject tissue corresponding to the different wavelength bands of received reflected light, a calculator for calculating a blood vessel characteristics amount in a subject tissue from the image data, a calculator for calculating an oxygen saturation level in the blood vessel from the image data, an image producer for producing a reference image of the subject tissue from the image data, an extractor for extracting a region of interest from the reference image, a producer for producing an enhanced image, and a display for displaying the enhanced image.

### BACKGROUND OF THE INVENTION

The present invention relates to an electronic endoscope system for acquiring information on a blood vessel from an image acquired by an electronic endoscope and producing an image from the acquired information.

In recent years, a number of diagnoses and treatments using electronic endoscopes have been made in the field of medicine. A typical electronic endoscope is equipped with an elongated insertion section that is inserted into a subject's body cavity. The insertion section has therein incorporated an imager such as a CCD at the tip thereof. The electronic endoscope is connected to a light source device, which emits light from the tip of the insertion section to illuminate the inside of a body cavity. With the inside of the body cavity illuminated by light, the subject tissue inside the body cavity is imaged by an imager provided at the tip of the insertion section. Images acquired by imaging undergoes various kinds of processing by a processor connected to the electronic endoscope before being displayed by a monitor. Thus, the electronic endoscope permits real-time observation of images showing the inside of the subject's body cavity and thus enables sure diagnoses.

The light source device uses a white light source such as a xenon lamp capable of emitting white broadband light whose wavelength ranges from a blue region to a red region. Use of white broadband light to illuminate the inside of a body cavity permits observing the whole subject tissue from the acquired images thereof. However, although images acquired by broadband light illumination permit generally observing the whole subject tissue, there are cases where such images fail to enable clear observation of subject tissues such as micro-blood vessels, deep-layer blood vessels, pit patters, and uneven surface profiles formed of recesses and bumps. As is known, such subject tissues may be made clearly observable when illuminated by narrowband light having a wavelength limited to a specific range. As is also known, image data obtained by illumination with narrowband light yields various kinds of information on a subject tissue such as oxygen saturation level in a blood vessel.

According to JP 6-285050 A, for example, the system therein described acquires a visible image (normal image) containing oxygen saturation level information and has separating means for separating a first and a second wavelength to acquire an image of a visible region containing oxygen saturation level information thereby to display an image where a variation of the oxygen saturation level is incorporated to the visible image.

### SUMMARY OF THE INVENTION

In recent years, there are demands for a system permitting diagnosis accompanied by simultaneous observation of blood vessel depth and oxygen saturation level. However, because of various causes including the absorbance of hemoglobin in blood vessels that vary significantly (see FIG. 5), simultaneously acquiring both blood vessel depth information and oxygen saturation level information is not an easy task.

According to JP 6-285050 A, for example, while provision of separating means for separating a first and a second wavelength enabled acquisition of information on the oxygen saturation level of hemoglobin, the visible image was changed only according to the variation of the oxygen saturation level, and the information on a blood vessel characteristics amount was not incorporated.

An object of the present invention is to provide an electronic endoscope system that comprises both blood vessel characteristics amount calculation means and oxygen saturation level calculation means and thus, with a combination of blood vessel characteristics amount and oxygen saturation level information, is capable of selectively enhancing and subduing a region of interest that is of interest in making a diagnosis.

To achieve the above object, the present invention provides an electronic endoscope system comprising a light source device for sequentially emitting light having different wavelength bands; an electronic endoscope for sequentially illuminating a subject tissue containing a blood vessel inside a body cavity with the light sequentially emitted from the light source device, receiving reflected light of the light from the subject tissue, and sequentially outputting image data of wavelength bands of the subject tissue corresponding to the different wavelength bands of the received reflected light; blood vessel characteristics amount calculating means for calculating a blood vessel characteristics amount containing at least one of a blood vessel depth, a blood vessel diameter, a blood vessel density, a blood vessel branch point density, and a fluorescent agent distribution in the subject tissue from the image data of the wavelength bands; oxygen saturation level calculation means for calculating an oxygen saturation level in the blood vessel of the subject tissue from the image data of the wavelength bands; image producing means for producing a reference image of the subject tissue from the image data of the wavelength bands; region-of-interest extraction means for extracting from the reference image a region of interest containing a predetermined blood vessel characteristics amount and a predetermined oxygen saturation level set by specifying information based on blood vessel characteristics amounts calculated in the subject tissue and oxygen saturation levels calculated in the blood vessel of the subject tissue; enhanced image producing means for producing an enhanced image in which the region of interest is enhanced in the reference image; and image displaying means for displaying the enhanced image.

Preferably, the blood vessel characteristics amount calculating means calculates the blood vessel depth as the blood vessel characteristics amount and the specifying information specifies that the blood vessel depth is 100 μm or less and the oxygen saturation level is 20% or less.

Preferably, the blood vessel characteristics amount calculating means calculates the blood vessel diameter as the blood vessel characteristics amount and the specifying information specifies that the blood vessel diameter is 20 μm or less and the oxygen saturation level is 20% or less.

Preferably, the blood vessel characteristics amount calculating means calculates the blood vessel density as the blood vessel characteristics amount and the specifying information specifies that the blood vessel density of blood vessels having a diameter of 20 μm or less is 2/(100 μm) or more and the oxygen saturation level is 20% or less.

Preferably, the blood vessel characteristics amount calculating means calculates the blood vessel branch point density as the blood vessel characteristics amount and the specifying information specifies that the blood vessel branch point density is 1/(50×50 (μm)²) or more and the oxygen saturation level is 20% or less.

Preferably, the blood vessel characteristics amount calculating means calculates a distribution of a luminance ratio of first pixel data corresponding to a first wavelength band and second pixel data corresponding to a second wavelength band that is different from the first wavelength band among the image data of the wavelength bands as the fluorescent agent distribution and the specifying information specifies that a size of the luminance ratio used as the fluorescent agent distribution is within a top 20% of the distribution of the luminance ratio and that the oxygen saturation level is 20% or less.

Preferably, the specifying information is set through input means and combinations of the blood vessel characteristics amount and the oxygen saturation level are previously set and the specifying information is set according to a combination selected through the input means.

According to the present invention, a first and a second narrowband signal corresponding to first and second narrowband light at least one of which has a central wavelength of 450 nm or less are acquired, blood vessel information containing both blood vessel depth information on a blood vessel depth and oxygen saturation level information on an oxygen saturation level are acquired based on the first and second narrowband signals thereof, and these information are displayed selectively or simultaneously on display means thereby to enable simultaneous acquisition of both the information on a blood vessel depth and the information on an oxygen saturation level and simultaneous display of both information.

### BRIEF DESCRIPTION OF DRAWINGS

FIG. 1 is an external view of an electronic endoscope system according to an embodiment of the invention.

FIG. 2 is a block diagram illustrating an electric configuration of the electronic endoscope system according to the embodiment of the invention.

FIG. 3 is a graph illustrating spectral transmittances of red, green, and blue filters.

FIG. 4A is a view for explaining operations of a CCD in a normal light image mode; FIG. 4B is a view for explaining operations of a CCD in a special light image mode.

FIG. 5 is a graph illustrating an absorption coefficient of hemoglobin.

FIG. 6 is a graph illustrating a correlation between first and second luminance ratios S1/S3 and S2/S3 on the one hand and blood vessel depth and oxygen saturation level on the other hand.

FIG. 7A is a view for explaining how a coordinate point (X*, Y*) in a luminance coordinate system is obtained from the first and the second luminance ratios S1*/S3* and S2*/S3*; FIG. 7B is a view for explaining how a coordinate point (U*, V*) in a blood vessel information coordinate system corresponding to the coordinate point (X*, Y*) is obtained.

FIG. 8 illustrates an image view of a screen given by a monitor displaying an enhanced broadband image and one of a blood vessel depth image and an oxygen saturation level image.

FIG. 9 illustrates an image view of a screen given by a monitor displaying an enhanced broadband image, a blood vessel depth image, and an oxygen saturation level image simultaneously.

FIG. 10 is a flow chart illustrating the first half of a procedure of calculating a blood vessel depth-oxygen saturation level information and extracting a region of interest based on a blood vessel characteristics amount and oxygen saturation level information to produce an enhanced image.

FIG. 11 is a flow chart illustrating the second half of a procedure of calculating a blood vessel depth-oxygen saturation level information and extracting a region of interest based on a blood vessel characteristics amount and oxygen saturation level information to produce an enhanced image.

### DETAILED DESCRIPTION OF THE INVENTION

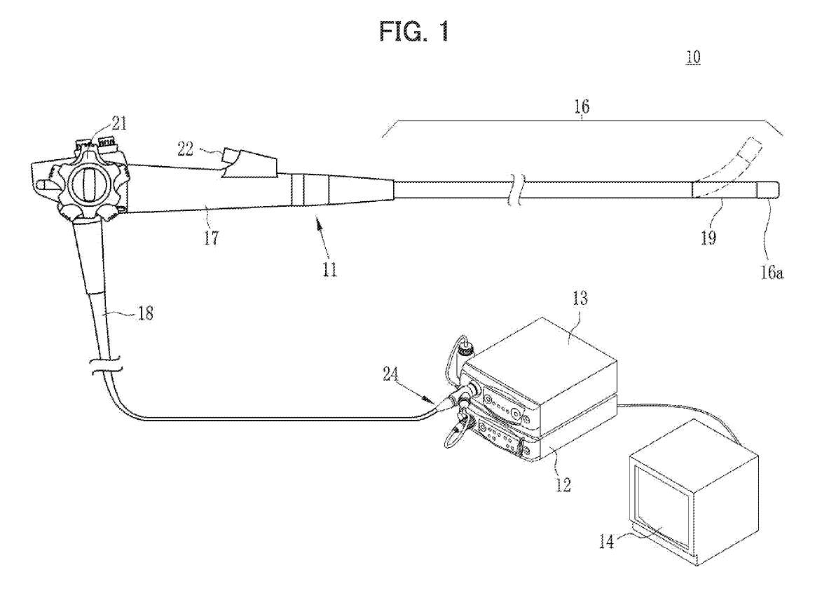

As illustrated in FIG. 1, an electronic endoscope system 10 comprises an electronic endoscope 11 for imaging the inside of a subject's body cavity, a processor 12 for producing an image of a subject tissue in the body cavity according to a signal acquired by imaging, a light source device 13 for supplying light for illuminating the inside of the body cavity, and a monitor 14 for displaying the image of the inside of the body cavity. The electronic endoscope 11 comprises a flexible insertion section 16 that is inserted into a body cavity, an operating section 17 provided at the base of the insertion section 16, and a universal cord 18 for connecting the operating section 17 to the processor 12 and the light source device 13.

The insertion section 16 has a bending portion 19 at the tip thereof comprising connected bending pieces. The bending portion 19 bends up and down, left and right in response to the operation of an angle knob 21 of the operating section 17. The bending portion 19 has at its tip a leading end portion 16a incorporating an optical system and other components for imaging the inside of a body cavity. The leading end portion 16a can be directed in a desired direction in the body cavity according to a bending operation of the bending portion 19.

The universal cord 18 has a connector 24 provided on the side thereof leading to the processor 12 and the light source device 13. The connector 24 is a composite type connector composed of a communication connector and a light source connector and removably connects the electronic endoscope 11 to the processor 12 and the light source device 13 through the connector 24.

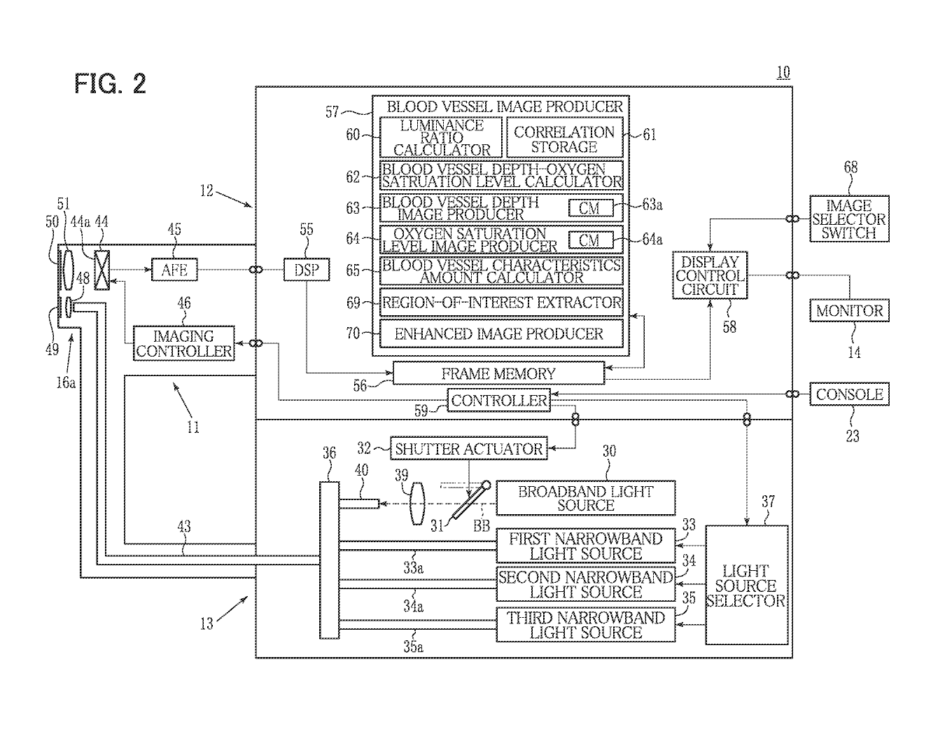

As illustrated in FIG. 2, the light source device 13 comprises a broadband light source 30, a shutter 31, a shutter actuator 32, first to third narrowband light sources 33 to 35, a coupler 36, and a light source selector 37. The broadband light source 30 is a xenon lamp, a white LED, a micro-white light source, or the like and produces broadband light BB having a wavelength ranging from a blue region to a red region (about 470 nm to 700 nm). The broadband light source 30 remains lighted at all times when the electronic endoscope 11 is in operation. The broadband light BB emitted from the broadband light source 30 is focused by a condenser lens 39 before entering a broadband optical fiber 40.

The shutter 31 is disposed between the broadband light source 30 and the condenser lens 39 so as to be movable between its inserted position where the shutter 31 is located on the optical path of the broadband light BB to block the broadband light BB and its retracted position where the shutter 31 is retracted from the inserted position to allow the broadband light BB to travel toward the condenser lens 39. The shutter actuator 32 is connected to a controller 59 in the processor to control the actuation of the shutter 31 according to an instruction from the controller 59.

The first to the third narrowband light sources 33 to 35 are laser diodes or the like. The first narrowband light source 33 produces narrowband light having a wavelength limited to 440 nm+/−10 nm, preferably 445 nm (referred to below as “first narrowband light N1”), the second narrowband light source 34 produces narrowband light having a wavelength limited to 470 nm+/−10 nm, preferably 473 nm (referred to below as “second narrowband light N2”), and the third narrowband light source 35 produces narrowband light having a wavelength limited to 400 nm+/−10 nm, preferably 405 nm (referred to below as “third narrowband light N3”). The first to the third narrowband light sources 33 to 35 are connected respectively to first to third narrowband optical fibers 33a to 35a, allowing the first to the third narrowband light N1 to N3 emitted by their respective light sources to enter the first to the third narrowband optical fibers 33a to 35a.

The coupler 36 connects a light guide 43 in the electronic endoscope to the broadband optical fiber 40, and the first to the third narrowband optical fibers 33a to 35a. Thus, the broadband light BB can enter the light guide 43 through the broadband optical fiber 40.

The first to the third narrowband light N1 to N3 can enter the light guide 43 through the first to the third narrowband optical fibers 33a to 35a.

The light source selector 37 is connected to the controller 59 in the processor and turns on or off the first to the third narrowband light sources 33 to 35 according to an instruction by the controller 59. According to the first embodiment, when the system is in the normal light image mode using the broadband light BB, the inside of a body cavity is illuminated by the broadband light BB to acquire a normal light image while the first to the third narrowband light sources 33 to 35 are turned off. In the special light image mode using the first to the third narrowband light N1 to N3, the illumination of the inside of the body cavity by the broadband light BB is terminated while the first to the third narrowband light sources 33 to 35 are sequentially turned on to acquire special light images.

Specifically, the light source selector 37 first turns on the first narrowband light source 33. Then, imaging of the subject tissue is started with the first narrowband light N1 illuminating the inside of the body cavity. Upon completion of imaging, the controller 59 gives a light source switching instruction to turn off the first narrowband light source 33 and turn on the second narrowband light source 34. Likewise, upon completion of imaging with the second narrowband light N2 illuminating the inside of the body cavity, the second narrowband light source 34 is turned off and the third narrowband light source 35 is turned on. Upon completion of imaging with the third narrowband light N3 illuminating the inside of the body cavity, the third narrowband light source 35 is turned off.

The electronic endoscope 11 comprises a light guide 43, a CCD 44, an analog processing circuit (AFE: analog front end) 45, and an imaging controller 46. The light guide 43 is a large-diameter optical fiber, a bundle fiber, or the like having its light-receiving end inserted in the coupler 36 in the light source device, whereas its light emitting end is directed toward an illumination lens 48 located in the leading end portion 16a. The light emitted by the light source device 13 is guided by the light guide 43 and emitted toward the illumination lens 48. The light admitted in the illumination lens 48 passes through an illumination window 49 attached to the end face of the leading end portion 16a to enter the body cavity. The broadband light BB and the first to the third narrowband light N1 to N3 reflected by the inside of the body cavity pass through an observation window 50 attached to the end face of the leading end portion 16a to enter a condenser lens 51.

The CCD 44 receives the light from the condenser lens 51 with its imaging surface 44a, performs photoelectric conversion of the received light to accumulate a signal charge, and reads out the accumulated signal charge as an imaging signal.

The read-out imaging signal is transmitted to an AFE 45. The CCD 44 is a color CCD whose imaging surface 44a has arranged therein three colors of pixels, red pixels, green pixels, and blue pixels, each provided with one of a red filter, a green filter, and a blue filter.

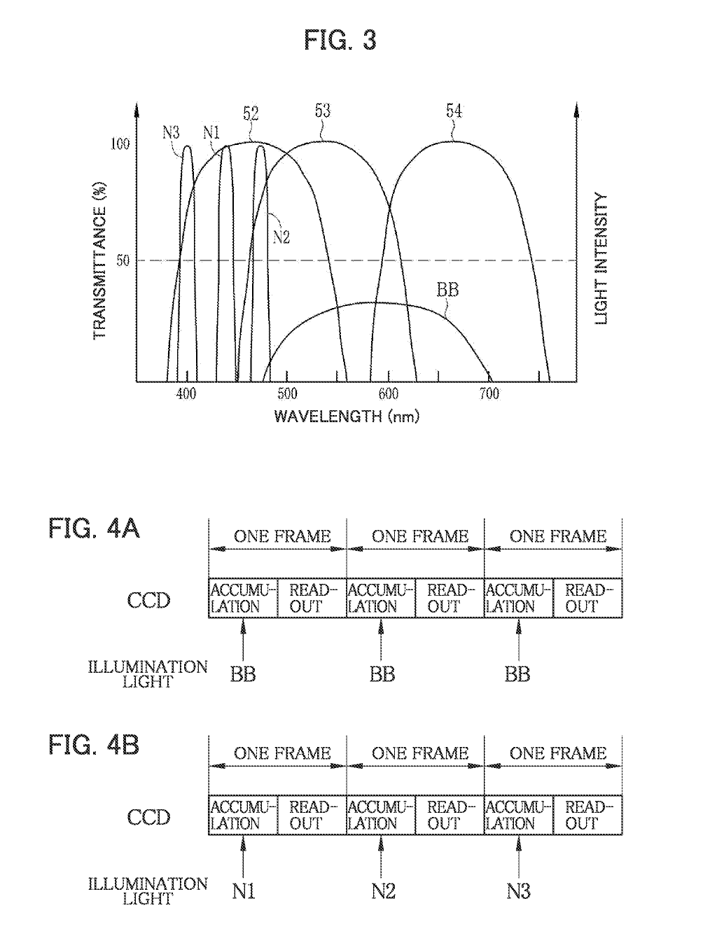

As illustrated in FIG. 3, the red filters, the green filters, and the blue filters have spectral transmittances 52, 53, and 54, respectively. Among the light entering the condenser lens 51, the broadband light BB has a wavelength ranging from about 470 nm to 700 nm. The red filters, the green filters, and the blue filters pass a wavelength range of broadband light BB corresponding to their spectral transmittances. Now, let imaging signal R be a signal photoelectrically converted by a red pixel, imaging signal G a signal photoelectrically converted by a green pixel, and imaging signal B a signal photoelectrically converted by a blue pixel. Then, the broadband light BB entering the CCD 44 gives a broadband imaging signal composed of the imaging signal R, the imaging signal G, and the imaging signal B.

Among the light entering the condenser lens 51, the first narrowband light N1 has a wavelength of 440 nm+/−10 nm and therefore passes through only the blue filters. Accordingly, the first narrowband light N1 entering the CCD 44 yields a first narrowband imaging signal composed of an imaging signal B. The second narrowband light N2 has a wavelength of 470 nm+/−10 nm and therefore passes through both the blue and green filters. Accordingly, the second narrowband light N2 entering the CCD 44 yields a second narrowband imaging signal composed of an imaging signal B and an imaging signal G. The third narrowband light N3 has a wavelength of 400 nm+/−10 nm and therefore passes through only the blue filters. Accordingly, the first narrowband light N3 entering the CCD 44 yields a third narrowband imaging signal composed of an imaging signal B.

The AFE 45 comprises a correlated double sampling circuit (CDS), an automatic gain control circuit (AGC), and an analog-to-digital converter (A/D) (none of them are shown). The CDS performs correlated double sampling of an imaging signal supplied from the CCD 44 to remove noise generated by actuation of the CCD 44. The AGC amplifies the imaging signal from which noise has been removed by the CDS. The analog-to-digital converter converts the imaging signal amplified by the AGC into a digital imaging signal having a given number of bits, which is applied to the processor 12.

The imaging controller 46 is connected to the controller 59 in the processor 12 and sends a drive signal to the CCD 44 in response to an instruction given by the controller 59. The CCD 44 outputs an imaging signal to the AFE 45 at a given frame rate according to the drive signal from the imaging controller 46. According to the first embodiment, when the system is in the normal light image mode, a total of two operations are performed in a one-frame acquisition period as illustrated in FIG. 4A: a step of accumulating a signal charge through photoelectric conversion of the broadband light BB and a step of reading out the accumulated signal charge as a broadband imaging signal. These operations are repeated throughout the normal light image mode.

By contrast, when the mode is switched from the normal light image mode to the special light image mode, a total of two operations are first performed in one frame of acquisition period as illustrated in FIG. 4B: a step of accumulating a signal charge through photoelectric conversion of the first narrowband light N1 and a step of reading out the accumulated signal charge as a first narrowband imaging signal. Upon completion of readout of the first narrowband imaging signal, a step of accumulating a signal charge through photoelectric conversion of the second narrowband light N2 and a step of reading out the accumulated signal charge as a second narrowband imaging signal are performed in one frame of acquisition period. Upon completion of readout of the second narrowband imaging signal, a step of accumulating a signal charge through photoelectric conversion of the third narrowband light N3 and a step of reading out the accumulated signal charge as a third narrowband imaging signal are performed in one frame of acquisition period.

As illustrated in FIG. 2, the processor 12 comprises a digital signal processor 55 (DSP), a frame memory 56, a blood vessel image producer 57, and a display control circuit 58, all of these components being controlled by the controller 59. The DSP 55 performs color separation, color interpolation, white balance adjustment, gamma correction, and the like of the broadband imaging signal and the first to the third narrowband imaging signals outputted from the AFE 45 of the electronic endoscope to produce broadband image data and the first to the third narrowband image data. The frame memory 56 stores the broadband image data and the first to the third narrowband image data produced by the DSP 55. The broadband image data is color image data containing colors of red, green, and blue.

The blood vessel image producer 57 comprises a luminance ratio calculator 60, a correlation storage 61, a blood vessel depth-oxygen saturation level calculator 62, a blood vessel depth image producer 63, an oxygen saturation level image producer 64, a blood vessel characteristics amount calculator, a region-of-interest extractor, and an enhanced image producer. The luminance ratio calculator 60 determines a blood vessel region containing a blood vessel from the first to the third narrowband image data stored in the frame memory 56. The luminance ratio calculator 60 obtains a first luminance ratio S1/S3 between the first and the third narrowband image data and a second luminance ratio S2/S3 between the second and the third narrowband image data corresponding to a pixel at the same position in the blood vessel region. S1 is a luminance of a pixel of the first narrowband image data, S2 a luminance of a pixel of the second narrowband image data, and S3 a luminance of a pixel of the third narrowband image data. The blood vessel region may be determined, for example, by a method whereby the blood vessel region is obtained from the difference between the luminance of a blood vessel of interest and the luminance of the other region.

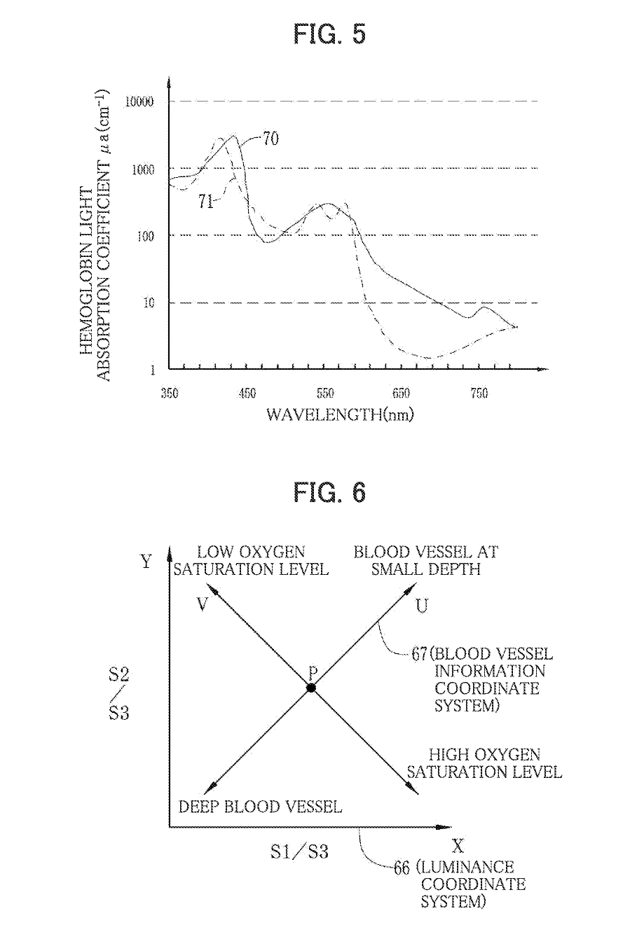

The correlation storage 61 stores a correlation between the first and the second luminance ratios S1/S3 and S2/S3 on the one hand and an oxygen saturation level in a blood vessel and a blood vessel depth on the other hand. That correlation is one where a blood vessel contains hemoglobin exhibiting light absorption coefficients as shown in FIG. 5 and is obtained by analyzing, for example, a number of the first to the third narrowband image data accumulated through diagnoses hitherto made. As illustrated in FIG. 5, the hemoglobin in a blood vessel has light absorptions characteristics having the light absorption coefficient μa changing according to the wavelength of light used for illumination. The light absorption coefficient μa indicates an absorbance or a degree of light absorption by hemoglobin and is a coefficient in an expression I₀exp(−μa×x) showing an attenuation of light by which hemoglobin was illuminated. In this expression, Io is the intensity of light emitted from the light source device to illuminate a subject tissue; x (cm) is a depth of a blood vessel inside the subject tissue.

A reduced hemoglobin 70 and an oxygenated hemoglobin 71 have different light absorption characteristics such that they have different absorbances except for the isosbestic points at which both exhibit the same absorbance (intersections of light absorption characteristics curves of hemoglobin 70 and 71 in FIG. 5). With a difference in absorbance, the luminance varies even when the same blood vessel is illuminated by light having the same intensity and the same wavelength. The luminance also varies when the illumination light has the same intensity but varies in wavelength because a difference in wavelength causes the light absorption coefficient μa to change.

In view of the light absorption characteristics of hemoglobin as described above and considering the fact that wavelengths whereby the absorbance varies according to the oxygen saturation level lie in a range of 445 nm and 504 nm and that light having a short wavelength and hence having a short reaching depth is required in order to retrieve blood vessel depth information, at least one of the first to the third narrowband light N1 to N3 preferably has a wavelength range whose central wavelength is 450 nm or less. According to the first embodiment of the invention, the first and the second narrowband light are such narrowband light. Further, with the same oxygen saturation level, a difference in wavelength causes a difference in absorption coefficient and also a difference in reaching depth into a mucus membrane. Therefore, using the property of light whose reaching depth varies with the wavelength permits obtaining correlation between luminance ratio and blood vessel depth.

As illustrated in FIG. 6, the correlation storage 61 stores a correlation in correspondence between the coordinate points in a luminance coordinate system 66 representing the first and the second luminance ratios S1/S3 and S2/S3 and the coordinate points in a blood vessel information coordinate system 67 representing oxygen saturation level and blood vessel depth. The luminance coordinate system 66 is an XY coordinate system, where the X axis shows the first luminance ratio S1/S3 and the Y axis shows the second luminance ratio S2/S3. The blood vessel information coordinate system 67 is a UV coordinate system provided on the luminance coordinate system 66, where the U axis shows the blood vessel depth and the V axis shows the oxygen saturation level. Because the blood vessel depth has a positive correlation with the luminance coordinate system 66, the U axis has a positive slope. The U axis shows that a blood vessel of interest is located at an increasingly smaller depth as a position on the U axis moves obliquely up rightward and that a blood vessel of interest is located at an increasingly greater depth as a position on the U axis moves obliquely down leftward. On the other hand, because the oxygen saturation level has a negative correlation with the luminance coordinate system 66, the V axis has a negative slope. The V axis shows that the oxygen saturation level is lower as a position on the V axis moves obliquely up leftward and that the oxygen saturation level is higher as a position on the V axis moves obliquely down rightward.

In the blood vessel information coordinate system 67, the U axis and the V axis cross each other at right angles at an intersection P. This is because the magnitude of absorbance reverses between illumination by the first narrowband light N1 and illumination by the second narrowband light N2. More specifically, as illustrated in FIG. 5, illumination by the first narrowband light N1 having a wavelength of 440 nm+/−10 nm allows the light absorption coefficient of the reduced hemoglobin 70 to be greater than that of the oxygenated hemoglobin 71 having a high oxygen saturation level whereas illumination by the second narrowband light N2 having a wavelength of 470 nm+/−10 nm allows the light absorption coefficient of the oxygenated hemoglobin 71 to be greater than that of the reduced hemoglobin 70 having a high oxygen saturation level, thus causing the magnitude of the absorbance to reverse.

When narrowband light permitting no absorbance reversal are used in lieu of the first to the third narrowband light N1 to N3, the U axis and the V axis do not cross each other at right angles. With illumination provided by the third narrowband light N3 having a wavelength of 400 nm+/−10 nm, the oxygenated hemoglobin and the reduced hemoglobin have a substantially equal light absorption coefficient.

The blood vessel depth-oxygen saturation level calculator 62 determines an oxygen saturation level and a blood vessel depth corresponding to the first and the second luminance ratios S1/S3 and S2/S3 calculated by the luminance ratio calculator 60 based on the correlation stored in the correlation storage 61. Now, in the first and the second luminance ratios S1/S3 and S2/S3 calculated by the luminance ratio calculator 60, let S1*/S3* and S2*/S3* be the first luminance ratio and the second luminance ratio respectively for a given pixel in the blood vessel region.

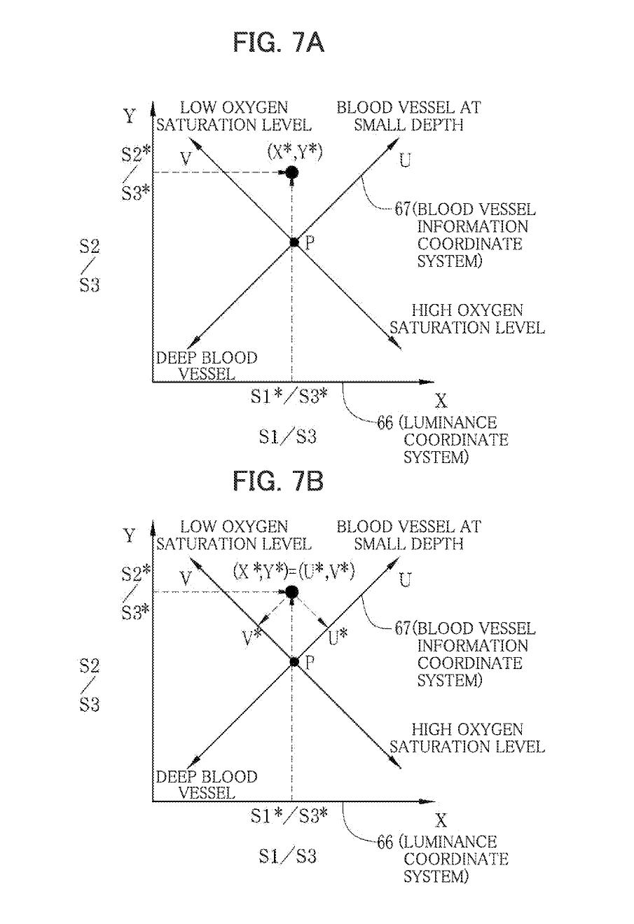

As illustrated in FIG. 7A, the blood vessel depth-oxygen saturation level calculator 62 determines a coordinate point (X*, Y*) corresponding to the first and the second luminance ratios S1*/S3* and S2*/S3* in the luminance coordinate system 66. Upon the coordinate point (X*, Y*) being determined, the blood vessel depth-oxygen saturation level calculator 62 determines a coordinate point (U*, V*) corresponding to the coordinate point (X*, Y*) in the blood vessel information coordinate system 67 as illustrated in FIG. 7B. Thus, blood vessel depth information U* and oxygen saturation level information V* are obtained for a given pixel in the blood region.

The blood vessel depth image producer 63 has a color map 63a (CM) where blood vessel depths are each assigned color information. More specifically, the color map 63a permits easy distinction of the blood vessel depth by color assignment such that, for example, a superficial-layer blood vessel is assigned a color of blue, an intermediate-layer blood vessel is assigned a color of green, and a deep-layer blood vessel is assigned a color of red. From the color map 63a, the blood vessel depth image producer 63 determines color information corresponding to the blood vessel depth information U* calculated by the blood vessel depth-oxygen saturation level calculator 62.

When all the pixels in the blood vessel region have been assigned color information, the blood vessel depth image producer 63 reads out broadband image data from the frame memory 56 and incorporates the color information in the read-out broadband image data. Thus, the blood vessel depth image data incorporating blood vessel depth information is produced. The blood vessel depth image data thus produced is stored again in the frame memory 56. The color information may be incorporated in one of the first to the third narrowband image data or in a synthesized image obtained by combining these in lieu of the broadband image data.

The oxygen saturation level image producer 64 has a color map 64a (CM) where oxygen saturation levels are assigned color information. More specifically, the color map 64a permits easy distinction of oxygen saturation level by color assignment such that, for example, a low oxygen saturation level is assigned a color of cyan, a medium oxygen saturation level is assigned a color of magenta, and a high oxygen saturation level is assigned a color of yellow. Similarly to the blood vessel depth image producer, the oxygen saturation level image producer 64 determines from the color map 64a color information corresponding to the oxygen saturation level information V* calculated by the blood vessel depth-oxygen saturation level calculator. Then, this color information is incorporated in the broadband image data to produce the oxygen saturation level image data. Like the blood vessel depth image data, the oxygen saturation level image data thus produced is stored in the frame memory 56.

The blood vessel characteristics amount calculator 65 calculates a blood vessel characteristics amount containing at least one of blood vessel depth (blood vessel depth from a subject tissue surface), blood vessel diameter, blood vessel density, blood vessel branch point density, and fluorescent agent distribution from specifying information entered through input means not shown. According to the first embodiment, while the blood vessel characteristics amount calculator 65 calculates the blood vessel depth as blood vessel characteristics amount, the blood vessel depth information U* is calculated, as described earlier, by the blood vessel depth-oxygen saturation level calculator 62. Thus, according to the first embodiment, the blood vessel characteristics amount calculator 65 corresponds to a part of the blood vessel depth-oxygen saturation level calculator 62 that produces the blood vessel depth information U*.

The region-of-interest extractor 69 extracts a region of interest containing a blood vessel characteristics amount and an oxygen saturation level corresponding to the specifying information on the blood vessel characteristics amount and the oxygen saturation level from the broadband image corresponding to the broadband image data based on the blood vessel characteristics amount and the oxygen saturation level information V*.

The specifying information designates information on a blood vessel characteristics amount and an oxygen saturation level in a region to be enhanced in display (i.e., region of interest) in an enhanced image produced by the enhanced image producer 70. The specifying information is entered by an endoscope operator or the like through input means (not shown). According to the first embodiment, when, for example, the specifying information designates a blood vessel depth of 100 μm or less and an oxygen saturation level of 20% or less, the region-of-interest extractor 69 extracts a region where the blood vessel depth is 100 μm or less and the oxygen saturation level is 20% or less as a region of interest from the broadband image.

Blood vessels lying in a depth of 100 μm or less are known to have a diameter of about 20 μm. It is empirically known that the third narrowband image data obtained by illumination with the third narrowband light N3 having a wavelength of 405 nm exhibits pixels corresponding to blood vessels having a diameter of about 10 μm to 20 μm lying in a depth of about 100 μm from the subject tissue surface in high contrast (great luminance value). Therefore, the enhanced image producer 70 is capable of extracting blood vessels corresponding to a depth of 100 μm or less and a diameter of 20 μm in terms of frequency band by extracting image data having a luminance value greater than a given threshold from the third narrowband image data.

The method whereby the region-of-interest extractor extracts a region of interest corresponding to the specifying information is not limited in any manner.

The enhanced image producer 70 produces an enhanced image where a region of interest is enhanced in a broadband image. Thus, the enhanced image data corresponding to the enhanced image is stored in the frame memory 56. According to the first embodiment, the enhanced image producer 69 produces an image where a region in which the blood vessel depth is 100 μm or less and the oxygen saturation level is 20% or less is enhanced (an image where the blood vessels corresponding to a diameter of about 20 μm in frequency band are enhanced).

The method whereby the enhanced display is achieved by the enhanced image producer 70 is not limited in any manner. For example, the luminance value may be increased or reduced; sharpness edge processing (edge enhancement) may be effected.

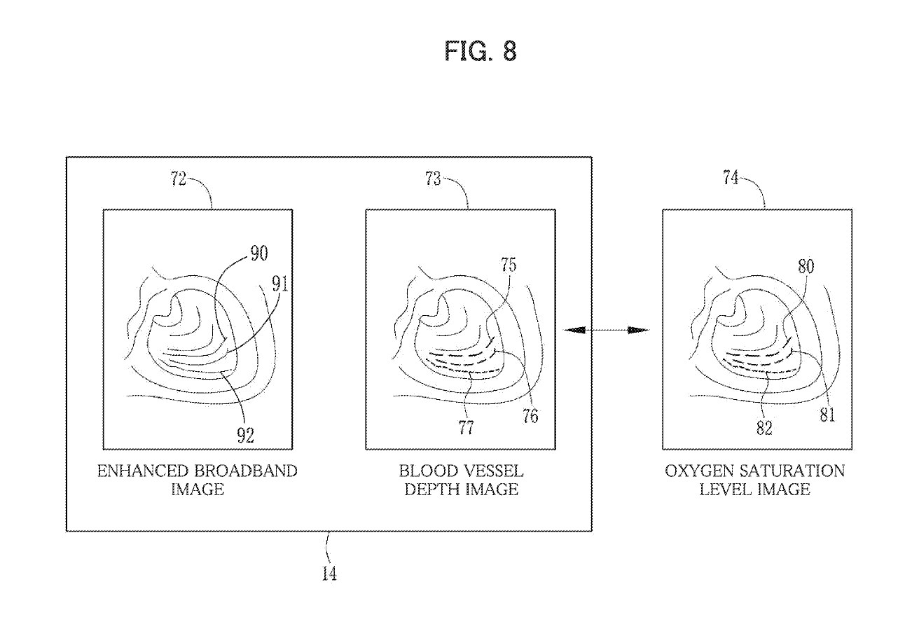

The display control circuit 58 reads out at least one enhanced image produced by the enhanced image producer 70 from the frame memory 56 and allows the monitor 14 to display the read-out image. The images may be displayed in various modes. As illustrated in FIG. 8, for example, the monitor 14 may display an enhanced broadband image 72 on one side and a blood vessel depth image 73 or an oxygen saturation level image 74 selected by an image selector switch 68 (see FIG. 2) on the other side. In the blood vessel depth image 73 illustrated in FIG. 8, a blood vessel image 75 is shown in blue indicating a superficial-layer blood vessel, a blood vessel image 76 is shown in green indicating an intermediate-layer blood vessel, and a blood vessel image 77 is shown in red indicating a deep-layer blood vessel. In the oxygen saturation level image 74, a blood vessel image 80 is shown in cyan indicating a lower oxygen saturation level, a blood vessel image 81 is shown in magenta indicating a medium oxygen saturation level, and a blood vessel image 82 is shown in yellow indicating a higher oxygen saturation level.

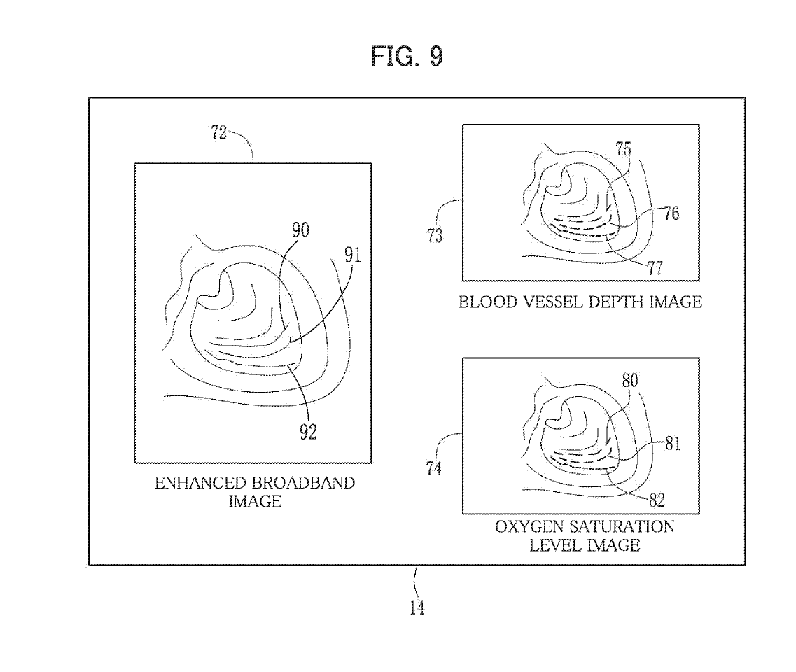

In contrast with the display mode shown in FIG. 8, the enhanced broadband image 72, the blood vessel depth image 73, and the oxygen saturation level image 74 may be displayed simultaneously as illustrated in FIG. 9.

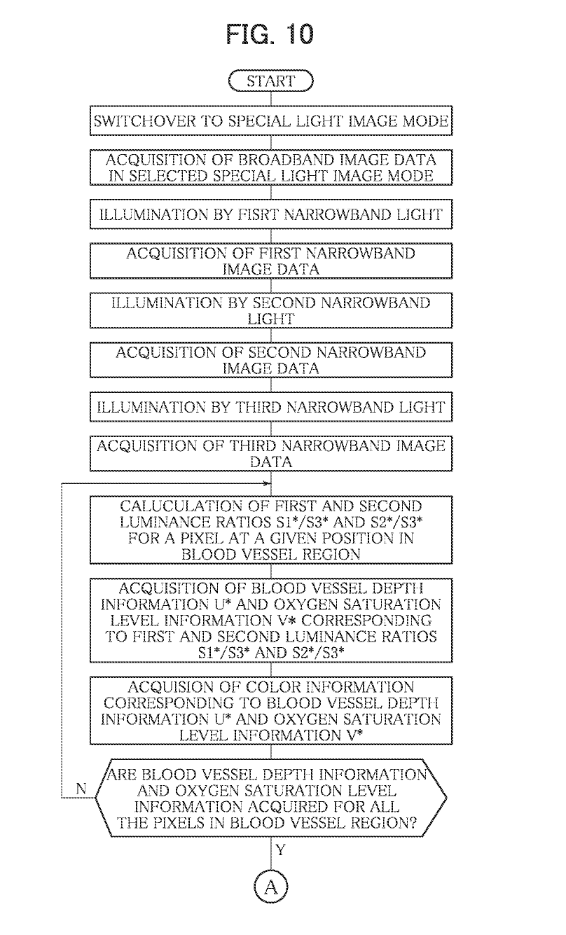

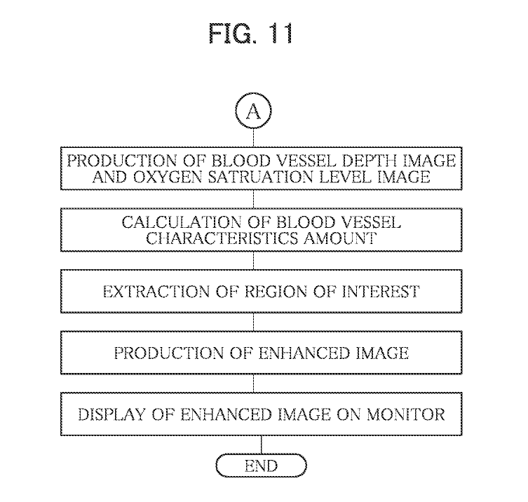

Next, the electronic endoscope system 10 will be described referring to the flowchart illustrated in FIG. 10.

First, the console 23 is operated to switch from the normal light image mode to the special light image mode. When the mode is switched to the special light image mode, the broadband image data as of the time the special light image mode is selected is stored in the frame memory 56 as image data used to produce the blood vessel depth image or the oxygen saturation level image. The broadband image data used to produce the blood vessel depth image or the like may be broadband image data as of the time the console is operated.

Upon receiving an illumination stop signal from the controller 59, the shutter actuator 32 moves the shutter 31 from the retracted position to the inserted position, causing the broadband light BB to stop illuminating the inside of the body cavity. When illumination by the broadband light BB is stopped, the controller 59 sends the light source selector 37 an illumination start instruction. Thereupon, the light source selector 37 turns on the first narrowband light source 33 to illuminate the inside of the body cavity with the first narrowband light N1. Upon the narrowband light N1 illuminating the inside of the body cavity, the controller 59 sends the imaging controller 46 an imaging instruction. Thus, imaging is done by illumination with the first narrowband light N1, and the first narrowband imaging signal obtained by the imaging is sent through the AFE 45 to the DSP 55. The DSP 55 produces the first narrowband image data based on the first narrowband imaging signal. The first narrowband image data thus produced is stored in the frame memory 56.

When the first narrowband image data has been stored in the frame memory 56, the light source selector 37 switches the light for illuminating the inside of the body cavity from the first narrowband light N1 to the second narrowband light N2 in response to the light source switching instruction from the controller 59. Then, imaging is done similarly to the case using the first narrowband light N1 to produce the second narrowband image data based on the second narrowband imaging signal obtained by the imaging. The second narrowband image data thus produced is stored in the frame memory 56.

When the second narrowband image data has been stored in the frame memory 56, the light source selector 37 switches the light for illuminating the inside of the body cavity from the second narrowband light N2 to the third narrowband light N3 in response to the light source switching instruction from the controller 59. Then, imaging is done similarly to the cases using the first and the second narrowband light N1 and N2 to produce the third narrowband image data based on the third narrowband imaging signal obtained by the imaging. The third narrowband image data thus produced is stored in the frame memory 56.

When the broadband image data and the first to the third narrowband image data have been stored in the frame memory 56, the luminance ratio calculator 60 determines the blood vessel region containing a blood vessel from three image data, i.e., the first narrowband image data, the second narrowband image data, and the third narrowband image data. Then, the luminance ratio calculator 60 calculates the first luminance ratio S1*/S3* between the first and the third narrowband image data and the second luminance ratio S2*/S3* between the second and the third narrowband image data corresponding to a pixel at the same position in the blood vessel region.

Next, the blood vessel depth-oxygen saturation level calculator 62 determines the coordinate point (X*, Y*) in the luminance coordinate system corresponding to the first and the second luminance ratios S1*/S3* and S2*/S3* based on the correlation stored in the correlation storage 61. Further, the coordinate point (U*, V*) in the blood vessel information coordinate system corresponding to the coordinate point (X*, Y*) is determined to obtain the blood vessel depth information U* and the oxygen saturation level information V* for a given pixel in the blood vessel region.

When the blood vessel depth information U* and the oxygen saturation level information V* have been obtained, color information corresponding to the blood vessel depth information U* is determined from the CM 63a in the blood vessel depth image producer while color information corresponding to the oxygen saturation level information V* is determined from the CM 64a in the oxygen saturation level image producer. The color information thus determined are stored in the RAM (not shown) in the processor 12.

Upon storage of the color information in the RAM, the above procedure is followed to obtain the blood vessel depth information U* and the oxygen saturation level information V* for all the pixels in the blood vessel region and determine color information corresponding to the blood vessel depth information U* and the oxygen saturation level information V*.

Then, when the blood vessel depth information, the oxygen saturation level information, and the corresponding color information have been obtained for all the pixels in the blood vessel region, the blood vessel depth image producer 63 reads out the broadband image data from the frame memory 56 and incorporates the color information stored in the RAM in the broadband image data to produce the blood vessel depth image data. The oxygen saturation level image producer 64 produces the oxygen saturation level image data as with the blood vessel depth image. The blood vessel depth image data and the oxygen saturation level image data thus produced are stored again in the frame memory 56.

Next, the blood vessel characteristics amount calculator 65 calculates the blood vessel characteristics amount based on the specifying information entered using input means not shown. The blood vessel characteristics amount is exemplified by blood vessel depth, blood vessel diameter, blood vessel density, blood vessel branch point density, and fluorescent agent, as mentioned earlier.

According to this embodiment, where the blood vessel depth is set as the blood vessel characteristics amount, the blood vessel characteristics amount calculator 65 calculates the blood vessel depth based on the specifying information on the blood vessel depth.

The specifying information includes information needed to calculate the blood vessel characteristics amount as mentioned earlier and is exemplified by information on a given blood vessel depth, a given blood vessel diameter, a given blood vessel density, a given blood vessel branch point density, and a given fluorescent agent distribution. According to this embodiment, blood vessel depth information is entered as the specifying information on the blood vessel characteristics amount. Similarly, information on a given oxygen saturation level also is entered as the specifying information. This is because both the blood vessel characteristics amount and the oxygen saturation level information are needed to extract a region of interest.

Suppose the specifying information is now given that the blood vessel depth as the blood vessel characteristics amount is 100 μm or less and the oxygen saturation level is 20% or less.

The region-of-interest extractor 69 extracts a region of interest containing a blood vessel characteristics amount (blood vessel depth in this embodiment) and an oxygen saturation level corresponding to the specifying information from the broadband image corresponding to the broadband image data based on the blood vessel characteristics amount and the oxygen saturation level information V* The given blood vessel depth and oxygen saturation level designated as threshold values may be selected as desired without limitation to the above examples.

Subsequent to the selection of a region of interest, region-of-interest extraction image data is produced and sent to the enhanced image producer 70.

The enhanced image producer 70 weights the region-of-interest extraction image data to produce the enhanced image data. The enhanced image data, with the region of interest extracted and enhanced for easy observation, enables high sensitivity observation of the region of interest when displayed on the monitor 14 or the like. The enhanced image data is stored in the frame memory 56.

The display control circuit 58 reads out the blood vessel depth image data, the oxygen saturation level image data, and the enhanced image data from the frame memory 56 and displays the enhanced broadband image 72, the blood vessel depth image 73, and the oxygen saturation level image 74 as illustrated in FIG. 8 or 9 on the monitor 14 based on the read-out image data. The monitor 14 illustrated in FIG. 8 simultaneously displays the enhanced broadband image 72 and one of the blood vessel depth image 73 and the oxygen saturation level image 74 in juxtaposition; the monitor 14 illustrated in FIG. 9 simultaneously displays the three images, i.e., the enhanced broadband image 72, the blood vessel depth image 73, and the oxygen saturation level image 74 in juxtaposition. The enhanced broadband image 72 displayed on the monitor 14 is a broadband image where a region in which the oxygen saturation level is 20% or less and the blood vessel depth is 100 μm or less is enhanced, which corresponds to the specifying information.

The first embodiment of the invention is as described above. The first embodiment enables a region where the blood vessels lie in a given blood vessel depth and have a given oxygen saturation level to be displayed with enhancement by calculating the blood vessel depth as blood vessel characteristics amount.

The image used when enhanced display is produced (reference image) is not limited to the broadband image; it may, for example, be an oxygen saturation level image corresponding to the oxygen saturation level image data, a blood vessel depth image corresponding to the blood vessel depth image data.

Next, a second embodiment of the invention will be described.

The electronic endoscope system according to the second embodiment of the invention is similar to the electronic endoscope system 10 according to the first embodiment except for the blood vessel characteristics amount calculator 65 and the region-of-interest extractor 69. Therefore, drawings and descriptions of the other features will not be provided below.

The second embodiment of the invention is different from the first embodiment in that the blood vessel diameter is used as the blood vessel characteristics amount.

According to this embodiment, where the blood vessel diameter is set as the blood vessel characteristics amount, the blood vessel characteristics amount calculator 65 calculates a region containing blood vessels having a given diameter. Examples of calculation of the blood vessel characteristics amount, specifically calculation of a blood vessel diameter region, include a calculation using a two-dimensional filter that extracts blood vessels having a given diameter.

Such a two-dimensional filter may be produced by estimating the distance between the leading end portion 16a of the endoscope and the subject and a magnification ratio, and obtaining a frequency in the image corresponding to the blood vessel diameter. The blood vessel diameter may be, for example, 20 μm or less for superficial-layer blood vessels. Next, a filter that enhances only that frequency band is designed in frequency space and then adapted to correspond to real space through Fourier transformation. In the present case, the filter characteristics need to be adjusted in frequency space so that the size of the filter can be contained within a realistic size of say about 5×5.

Application of the two-dimensional filter thus produced to the broadband image data permits extraction of blood vessels having a given blood vessel diameter.

Suppose the specifying information is now given that the blood vessel diameter as the blood vessel characteristics amount is 20 μm or less and the oxygen saturation level is 20% or less.

The blood vessel characteristics amount calculator 65 extracts blood vessels corresponding to the specifying information using the two-dimensional filter for calculating a region containing blood vessels having a diameter designated by the specifying information. The given blood vessel diameter and the oxygen saturation level designated as the threshold values may be selected as desired without limitation to the above examples.

The region-of-interest extractor 69 extracts as a region of interest a region where the blood vessel diameter is in a range of 20 μm or less and the oxygen saturation level is in a range of 20% or less from the broadband image corresponding to the broadband image data corresponding to the specifying information. The image data is not limited to the broadband image data and may be any of the blood vessel depth image data and the oxygen saturation level image data. This embodiment shares the procedure to follow with the first embodiment. Specifically, the enhanced broadband image 72 displayed on the monitor 14 represents, for example, a region where the blood density is in a range of 20 μm or less and the oxygen saturation level is in a range of 20% or less.

The method of extracting a region of interest based on the blood vessel diameter is not limited to the method described above and may be any of various known methods.

Next, a third embodiment of the invention will be described.

The electronic endoscope system according to the third embodiment of the invention is similar to the electronic endoscope system 10 according to the first embodiment except for the blood vessel characteristics amount calculating means 65 and the region-of-interest setting means 69. Therefore, drawings and descriptions of the other features will not be provided below.

The third embodiment of the invention is different from the first embodiment in that the blood vessel density is used as the blood vessel characteristics amount.

According to this embodiment, where the blood vessel density is set as the blood vessel characteristics amount, the blood vessel characteristics amount calculator 65 calculates the blood vessel density based on the specifying information on the blood vessel density.

The blood vessel characteristics amount calculator 65 acquires one of the first to the third narrowband image data stored in the frame memory 56. Because this embodiment uses the superficial blood vessel density as reference, a portion having a high blood vessel density, for example, is extracted from the first narrowband image data. The extraction of the portion having a high blood vessel density is achieved by binarizing the first narrowband image data. The first narrowband image data is binarized by assigning a pixel value of 1 to the pixels of a blood vessel and a pixel value of 0 to the other pixels in that image data. A threshold value used in assignment of 1 and 0 may for example be a mean value of the pixel values of the first narrowband image data.

The region-of-interest extractor 69 judges whether each pixel in the binarized image data binarized by the above method is a region having a blood vessel density corresponding to the specifying information. When the proportion of white pixels in a given square region centering on that particular pixel in the blood vessel density region is greater than a given threshold value, that pixel is judged to be a blood vessel density region corresponding to the specifying information Preferably, the given threshold value is for example about 30% and the size of the square is for example about a thousandth of the whole image.

Suppose the specifying information is now given that the blood vessel density as the blood vessel characteristics amount is 2/(100 μm) or more and the oxygen saturation level is 20% or less.

The blood vessel characteristics amount calculator 69 judges whether each pixel has a blood vessel density corresponding to the specifying information using the blood vessel density designated by the specifying information as threshold value. The given blood vessel density and oxygen saturation level designated as threshold values may be selected as desired without limitation to the above examples.

Specifically, the region-of-interest extractor 69 extracts as a region of interest a region where the blood vessel density is in a range of 2/(100 μm) or more and the oxygen saturation level is in a range of 20% or less from the broadband image corresponding to the broadband image data corresponding to the specifying information. The image data is not limited to the broadband image data and may be any of the blood vessel depth image data and the oxygen saturation level image data. This embodiment shares the procedure to follow with the first embodiment. Specifically, the enhanced broadband image displayed on the monitor 14 represents, for example, a region where the blood density is in a range of 2/(100 μm) or more and the oxygen saturation level is in a range of 20% or less.

The method of setting a blood vessel density is not limited to the method described above and may be any of various known methods.

Judgment as to the blood vessel density set as the blood vessel characteristics corresponding to the specifying information may be made with respect to a reference of a region containing blood vessels having a diameter of 20 μm or less with a density of 2/(100 μm) or more in lieu of the reference used in the above example.

Next, a fourth embodiment of the invention will be described.

The fourth embodiment of the invention is the same as the first embodiment except that the blood vessel branch point density is used as the blood vessel characteristics amount.

The electronic endoscope system according to the fourth embodiment of the invention is similar to the electronic endoscope system 10 according to the first embodiment except for the blood vessel characteristics amount calculator 65 and the region-of-interest extractor 69. Therefore, drawings and descriptions of the other features will not be provided below.

According to the fourth embodiment of the invention, the blood vessel characteristics amount calculator 65 sets the blood vessel branch point density as the blood vessel characteristics amount in the acquired image data.

According to this embodiment, where the blood vessel branch point density is set as the blood vessel characteristics amount, the blood vessel characteristics amount calculator 65 calculates the blood vessel branch point density based on the specifying information on the blood vessel branch point density, whereupon the region-of-interest extractor 69 extracts a region of interest corresponding to the blood vessel branch point density region corresponding to the specifying information.

First, the blood vessel characteristics amount calculator 65 acquires one of the first to the third narrowband image data stored in the frame memory 56. Because this embodiment uses the blood vessel branch point density in the superficial layer as reference, a portion having a high blood vessel branch point density is extracted from the first narrowband image data. The extraction of the portion having a high blood vessel branch point density may be achieved by binarizing the first narrowband image data as in the third embodiment to search for branch points in the binarized first narrowband image data by a template matching method. That is, a small, V-shaped, reference binarized image representing a blood vessel branch point is produced to search for points where the difference from that reference image is not greater than a given threshold value.

Because blood vessels bifurcate in various directions and at various angles, reference images having a plurality of patterns need to be produced. The region-of-interest extractor 69 judges whether each pixel has a blood vessel branch point density corresponding to the specifying information by the same method as used in the third embodiment and performs extraction.

Suppose the specifying information is now given that the blood vessel branch point density as the blood vessel characteristics amount is 1/(50×50 (μm)²) or more.

The blood vessel characteristics amount calculator 69 judges whether each region has a blood vessel branch point density corresponding to the specifying information using the blood vessel branch point density designated by the specifying information as threshold value. The given blood vessel branch point density and oxygen saturation level designated as threshold values may be selected as desired without limitation to the above examples.

Specifically, the region-of-interest extractor 69 extracts as a region of interest a region where the blood vessel branch point density is in a range of 1/(50×50 (μm)²) or more and the oxygen saturation level is in a range of 20% or less from the broadband image corresponding to the broadband image data corresponding to the specifying information. The image data is not limited to the broadband image data and may be any of the blood vessel depth image data and the oxygen saturation level image data. This embodiment shares the procedure to follow with the first embodiment. Specifically, the enhanced broadband image 72 displayed on the monitor 14 represents, for example, a region where the blood vessel branch point density is in a range of 1/(50×50 (μm)²) or more and the oxygen saturation level is in a range of 20% or less.

The method of setting the blood vessel branch point density is not limited to the method described above and may be any of various known methods.

According to a fifth embodiment of the invention, a blood vessel characteristics amount calculator 65 sets a fluorescent agent distribution as the blood vessel characteristics amount in the acquired image data. The fluorescent agent mentioned here, which may be, for example, ICG (indocyanine green), is distributed in blood vessels by intravenous injection prior to image acquisition. The fluorescent agent distribution, when the IGC is used, is calculated as the luminance of pixels as imaged using near-infrared light (e.g. about 730 nm).

Accordingly, this embodiment comprises a fourth narrowband light source for emitting near-infrared light 13. Subsequent to the distribution of the fluorescent agent in blood vessels, a fourth narrowband image (near-infrared light image) is acquired using fourth narrowband light N4. Being near-infrared light, the fourth narrowband light is passed through the red filters and undergoes photoelectric conversion through the red pixels of the CCD 44, so that the fourth narrowband image data is stored in the frame memory 56 as the imaging signal R.

The fourth narrowband light source is, for example, a light source such as a laser diode that permits easy light amount variation through light intensity modulation, pulse width modulation, or the like and configured and operated similarly to the first to the third narrowband light sources 33 to 35.

According to this embodiment, where the fluorescent agent distribution is set as the blood vessel characteristics amount, a region is calculated where the fluorescent agent distribution is such that the luminance ratio is within a given upper range of the distribution of the third luminance ratio S4/S3 based on the specifying information on the third luminance ratio S4/S3 between the third and the fourth narrowband image data. S3 is the luminance of a pixel of the third narrowband image data; S4 is the luminance of a pixel of the fourth narrowband image data. According to this embodiment, the blood vessel characteristics amount calculator 65 calculates the distribution of the third luminance ratio S4/S3 as fluorescent agent distribution while the third luminance ratio S4/S3 is calculated by the luminance ratio calculator 60. According to the fifth embodiment of the invention, therefore, a part of the blood vessel characteristics amount calculator 65 corresponds to the luminance ratio calculator 60 that calculates the third luminance ratio S4/S3. The narrowband image data is not limited to the third narrowband image data and may be the first narrowband image data or the second narrowband image data, provided that comparison with the fourth narrowband image data is possible.

As described above, the blood vessel characteristics amount calculator 65 also performs the function of the luminance ratio calculator 60 according to this embodiment. First, the luminance ratio calculator 60, i.e., the blood vessel characteristics amount calculator 65 according to this embodiment, obtains the third luminance ratio S4/S3, which is a luminance ratio between the third and the fourth narrowband image data for the pixel in the same position in the blood vessel region. According to this embodiment, the blood vessel characteristics amount calculator 65 calculates the distribution (histogram) of the third luminance ratio S4/S3 by performing statistical processing on the occurrence rates of the luminance ratios for all the pixels in the blood vessel.

Suppose the specifying information is now given that the luminance ratio is within the top 20% of the distribution of the third luminance ratio S4/S3 as the fluorescent agent distribution, which is the blood vessel characteristics amount, and that the oxygen saturation level is 20% or less.

The region-of-interest extractor 69 judges whether each pixel is in the fluorescent agent distribution corresponding to the specifying information using the luminance ratio designated by the specifying information as threshold value. The given luminance ratio and oxygen saturation level designated as threshold values may be selected as desired without limitation to the above examples.

Thus, according to the specifying information, the region-of-interest extractor 69 extracts, as a region of interest, a region where the luminance ratio is within the top 20% of the distribution of the third luminance ratio S4/S3 as the fluorescent agent distribution and where the oxygen saturation level is in a range of 20% or less from the broadband image corresponding to the broadband image data. The image data is not limited to the broadband image data and may be the blood vessel depth image data or the oxygen saturation level image data. This embodiment shares the procedure to follow with the first embodiment. Specifically, the enhanced broadband image 72 displayed on the monitor 14 represents, for example, a region where the luminance ratio is within the top 20% of the distribution of the third luminance ratio S4/S3 as the fluorescent agent distribution and where the oxygen saturation level is in a range of 20% or less.

The method of setting the blood vessel branch point density is not limited to the method described above and may be any of various known methods.

Next, a sixth embodiment of the invention will be described.

The sixth embodiment is different from the first embodiment in that there are previously set a plurality of combinations of the blood vessel characteristics amount and the oxygen saturation level information and the specifying information is set according to a combination selected through the input means.

The combinations are stored, for example, in the form of a table in the blood vessel image producer 57 and selected by a selector switch, which is not shown.

TABLE 1
Combination Table

| Com- | Oxygen | Blood vessel | Enhan- | Enhance- |
|---|---|---|---|---|
| bina- | saturation | characteristics | ced | ment |
| tion | level | amount | region | level |
| A | 20% or | Depth 100 μm or less | diameter of | 1 |
| | less | | about | |
| | | | 20 μm | |
| B | 20% or | Diameter 20 μm or less | — | 4 |
| | less | | | |
| C | 20% or | Density 2/(100 μm) or more | — | 3 |
| | less | | | |
| D | 20% or | Branch point density 1/ | — | 2 |
| | less | (50 × 50 (μm)²) or more | | |
| E | 20% or | Fluorescent agent distribution | — | 5 |
| | less | (luminance ratio) | | |
| | | within top 20% | | |
| F | — | — | — | — |

An example of the combination table is shown in Table 1. The observer can select a combination of A to F using input means not shown. The enhancement level indicates a weighting level in the region of interest.

Upon selection of one from the above combinations, the blood vessel characteristics amount is calculated, the region of interest corresponding to the specifying information is extracted and enhanced in any of the broadband image data, the blood vessel depth image data, and the oxygen saturation level image data based on the blood vessel characteristics amount, the oxygen saturation level information, etc. corresponding to the above combination selected from the combination table given in Table 1, enhanced image data is stored in the frame memory 56, and displayed, where necessary, on the monitor 14 or the like as the enhanced broadband image 72. The enhanced broadband image 72 displayed on the monitor 14 is a broadband image where a region corresponding to the blood vessel characteristics amount and the oxygen saturation level described in combinations of A to E in Table 1 is enhanced. The combinations A to E in Table 1 correspond to the first to the fifth embodiments.

The procedure to follow is the same as with the electronic endoscope system 10 according to the first and other embodiments.

The combinations are not limited to those given in Table 1 and may be determined as desired.

The present invention is basically as described above. The present invention is not limited to any of the embodiments described above and permits various modifications without departing from the spirit of the present invention.

What is claimed is:

1. An electronic endoscope system, comprising:
a light source device for sequentially emitting light having different wavelength bands;
an electronic endoscope for sequentially illuminating a subject tissue containing a blood vessel inside a body cavity with said light sequentially emitted from said light source device, receiving reflected light of said light from said subject tissue, and sequentially outputting image data of wavelength bands of said subject tissue corresponding to the different wavelength bands of said received reflected light;
blood vessel characteristics amount calculating means for calculating a blood vessel characteristics amount containing at least one of a blood vessel depth, a blood vessel diameter, a blood vessel density, a blood vessel branch point density, and a fluorescent agent distribution in said subject tissue from said image data of the wavelength bands;
oxygen saturation level calculation means for calculating an oxygen saturation level in said blood vessel of said subject tissue from said image data of the wavelength bands;
image producing means for producing a reference image of said subject tissue from said image data of the wavelength bands;
region-of-interest extraction means for extracting from said reference image a region of interest containing a predetermined blood vessel characteristics amount and a predetermined oxygen saturation level set by specifying information based on blood vessel characteristics amounts calculated in said subject tissue and oxygen saturation levels calculated in said blood vessel of said subject tissue;
enhanced image producing means for producing an enhanced image in which said region of interest is enhanced in said reference image; and
image displaying means for displaying said enhanced image.

2. The electronic endoscope system according to claim 1, wherein said blood vessel characteristics amount calculating means calculates said blood vessel depth as said blood vessel characteristics amount.

3. The electronic endoscope system according to claim 2, wherein said specifying information specifies that said blood vessel depth is 100 μm or less and said oxygen saturation level is 20% or less.

4. The electronic endoscope system according to claim 1, wherein said blood vessel characteristics amount calculating means calculates said blood vessel diameter as said blood vessel characteristics amount.

5. The electronic endoscope system according to claim 4, wherein said specifying information specifies that said blood vessel diameter is 20 μm or less and said oxygen saturation level is 20% or less.

6. The electronic endoscope system according to claim 1, wherein said blood vessel characteristics amount calculating means calculates said blood vessel density as said blood vessel characteristics amount.

7. The electronic endoscope system according to claim 6, wherein said specifying information specifies that said blood vessel density of blood vessels having a diameter of 20 μm or less is 2/(100 μm) or more and said oxygen saturation level is 20% or less.

8. The electronic endoscope system according to claim 1, wherein said blood vessel characteristics amount calculating means calculates said blood vessel branch point density as said blood vessel characteristics amount.

9. The electronic endoscope system according to claim 8, wherein said specifying information specifies that said blood vessel branch point density is 1/(50×50 (μm)²) or more and said oxygen saturation level is 20% or less.

10. The electronic endoscope system according to claim 1, wherein said blood vessel characteristics amount calculating means calculates a distribution of a luminance ratio of first pixel data corresponding to a first wavelength band and second pixel data corresponding to a second wavelength band that is different from said first wavelength band among said image data of the wavelength bands as said fluorescent agent distribution.

11. The electronic endoscope system according to claim 10, wherein said specifying information specifies that a size of said luminance ratio used as said fluorescent agent distribution is within a top 20% of said distribution of said luminance ratio and that said oxygen saturation level is 20% or less.

12. The electronic endoscope system according to claim 1, wherein said specifying information is set through input means.

13. The electronic endoscope system according to claim 1, wherein combinations of said blood vessel characteristics amount and said oxygen saturation level are previously set and said specifying information is set according to a combination selected through said input means.
